(19) United States
(12) Patent Application Publication
(10) Pub. No.: US 2007/0110781 A1
(43) Pub. Date: May 17, 2007

(54) ANTIMICROBIAL ARTICLE FOR USE IN MEDICAL ENVIRONMENT

(75) Inventors: Thomas W. Kotterer, Greensboro, NC (US); Clifford D. Weston, (US)

Correspondence Address: Cliff D. Weston, Suite 125, 11515 Vanstory Drive, Huntersville, NC, 28078, US

(73) Assignee: Microban Products Company, Huntersville, NC (US)

(21) Appl. No.: 11/559,895

(22) Filed: Nov. 14, 2006

Related U.S. Application Data
(60) Provisional application No. 60/736,440, filed on Nov. 14, 2005.

(51) Int. Cl. A01N 25/00; A47B 71/00

(52) U.S. Cl. 424/405; 5/600

(57) ABSTRACT

A medical bed article includes one or more components having an antimicrobial agent disposed therein or affixed thereto. Components can include a headboard, footboard, side rail, or other frame member, as well as cladding on such member. The bed components can be constructed of a metal or a polymeric material.

### CROSS-REFERENCE TO RELATED APPLICATION

This application claims priority to U.S. Ser. No. 60/736,440, filed on Nov. 14, 2005, and which is incorporated herein.

### BACKGROUND OF THE INVENTION

The invention relates to the field of health care accessories, and more specifically to a medical bed suitable for use in a healthcare setting which has imparted therein built-in and long lasting antimicrobial characteristics.

One of the challenges faced in built-in antimicrobial technology is selecting an efficacious antimicrobial agent for a particular product. For example, one antimicrobial agent may work well in interior applications (e.g., interior paint) yet be unsuitable for some outdoor applications (e.g., house siding). Similarly, an agent that works well against one class of microbe (e.g., fungi) may not work against another (e.g., bacteria). One likewise may anticipate that a combination of a particular bactericide and fungicide will work to provide the degree of antimicrobial protection sought for a particular product, only to discover that the two active agents are incompatible or must be delivered to the product separately and thus more expensively.

Equally challenging is incorporation of an antimicrobial agent into a material, such as a polymer resin, rubber compound, or the like, without adversely changing the physical or other properties of the material or altering the efficacy of the antimicrobial agent. The chemical milieu in which the antimicrobial agent will reside, the use-life conditions it will face, and the processes under which the article will be manufactured each can reduce or destroy the antimicrobial characteristic of the agent. Conversely, the specific chemical nature of the antimicrobial agent may perturb the molecular structure of the article or its fabrication.

Accordingly, imparting antimicrobial characteristics to particular products is not simply a matter of pulling an antimicrobial agent off of a shelf and adding it to an existing product. Many variables must be considered, and sometimes a commercially acceptable solution (i.e., effective and economically acceptable) is not readily apparent. Furthermore, as the field of built-in antimicrobial protection grows, each new product presents researchers with a new set of problems.

Therefore, a continuing need exists for new antimicrobial compositions that can be added to the arsenal of weapons used to fight the proliferation of microbes on consumer and industrial products.

### DETAILED DESCRIPTION OF PREFERRED EMBODIMENT(S)

As used herein, the terms “microbe” or “microbial” should be interpreted to encompass any of the microscopic organisms commonly studied by microbiologists. Such organisms include, but are not limited to, bacteria and fungi as well as other single-celled organisms such as mold, mildew and algae. Viral particles and other infectious agents are also included in the term microbe.

The term “antimicrobial” includes biostatic activity, i.e., where the proliferation of microbiological species is reduced or eliminated, and true biocidal activity where microbiological species are killed. For ease of discussion, this detailed description may make reference to bacteria and antibacterial agents. This method of presentation should not be interpreted as limiting the scope of the invention in any way.

The term efficacy, as used herein, is defined as the characteristic of inhibiting the growth and/or proliferation of a microbe on a substrate.

The term non-metallic as used herein means antimicrobial agents, other than quaternary ammonium compounds, that do not contain or utilize metal ions such as, for example, silver or copper.

An article for use in a clinical or hospital setting can include, without limitation, beds and bed frame members, bedside trays (free trays or standing trays), chairs, tables, counters, carts, cabinets and other storage-related furniture, storage cases, drawer pulls and handles, doors and door handles, floor mats, window treatments and control mechanisms therefor (e.g., a rotatable rod for adjustment of horizontal mini-blinds), and the like.

In more detail, a bed can include, without limitation, elements such as a headboard, footboard, and side rails (including protective side rails); supportive frame members; legs and bed under-structures; handles and grips; motorized bed controller housings, face panels, control panels; and the like. As used herein, a bed can include a gurney, stretcher, spine board or other article designed for transport of a person.

Headboard, footboard, side rails, and other components of a medical bed conventionally are constructed of metal, with polymeric pieces or cladding affixed thereto. Exemplary polymeric cladding articles can be made of acrylonitrile butadiene styrene (“ABS”) and olefin cladding. Exemplary olefins include, by way of illustration, polypropylene and polyethylene. Other polymeric substances can be employed.

Alternatively, some bed elements can be manufactured of a metal having a powder coating applied thereon. Compositions and methods for the inclusion of an antimicrobial agent in a coating powder are known to skilled artisans, such as disclosed in U.S. Pat. No. 5,238,749.

The antimicrobial agents useful with the above polymeric materials include 2,4,4′-trichloro-2-hydroxydiphenyl ether (i.e., triclosan) or other substituted or unsubstituted phenol; quaternary ammonium compounds, gentian violet compounds, biguanide compounds, iodine compounds, and mixtures thereof.

Examplary quaternary ammonium compounds include, without limitation: alkyl-dimethyl-benzylammonium chloride, alkyl-dimethyl-ethylbenzylammonium chloride, dodecyl-dimethyl-3,4-dichlorobenzylammonium chloride, dodecyl-di-(2-hydroxyethyl)-benzylammonium chloride, dodecyl-di-(2-hydroxyethyl)-benzylammonium pentachlorophenolate, dodecyl-di-(2-hydroxyethyl)-benzylammonium 4-methylbenzoate, dodecyl-dimethyl-phenoxyethylammonium bromide, 4-diisobutyl-phenoxyethoxyethyl-dimethylbenzylammonium chloride, 4-diisobutyl-cresoxyethoxyethyl-dimethylbenzylammonium chloride, dimethyl-didecylammonium chloride, cetyl-trimethylammonium bromide, dodecyl-pyridinium chloride, cetyl pyridinium chloride, dodecyl-isoquinolinium chloride, decamethylene-bis-4-aminoquinaldinium dichloride, alpha-(p-tolyl)-dodecyl-trimethyl-ammonium methosulphate, (dodecanoyl-N-methylaminoethyl)-(phenylcarbamoylmethyl)-dimethylammonium chloride, cetyl pyridinium chloride (1-hexadecylpyridinium chloride), and mixtures thereof.

A non-exhaustive list of gentian violet compounds include gentian violet and dimethyl gentian violet.

Exemplary substituted or unsubstituted phenols include, without limitation: phenol, mono- and poly-chlorophenols, cresols, 4-chloro-3-methylphenol, 3,5-dimethyl-4-chlorophenol, thymol, 4-chlorothymol, 4-t-amylphenol, saligenin, 4-n-hexylresorcinol, carvacrol, 2-phenylphenol, 2-benzyl-4-chlorophenol, 2,2′-dihydroxy-5,5′-dichlorodiphenylmethane, 2,2′-dihydroxy-3,3′,5,5′,6,6′-hexachlorodiphenylmethane, 2,2′-dihydroxy-5,5′-dichlorodiphenyl sulphide, 2,2′-dihydroxy-3,3′,5,5′-tetrachlorodiphenyl sulphide, 2-hydroxy-2′,4,4′-trichlorodiphenyl ether, dibromosalicyl, 3,5-dimethyl-4-chlorophenol (chloroxylenol), thymol (isopropyl-m-cresol), and mixtures thereof.

Non-limiting iodine compounds can include: potassium iodate, potassium iodide, iodine complexes (such as KI:I2 and polyvinylpyrrolidone:iodine) and iodine (herein considered an “iodine compound”).

The identity and amount of the particular antimicrobial agent(s) used can vary depending on the particular material employed. In some cases, the amount and/or type of the antimicrobial agent may vary depending on the type and amount of contamination which is likely to occur. The specific level depends on the antimicrobial agent(s) selected and the polymeric material to be treated. Generally speaking, it has been found that an antimicrobial agent concentration in the range of from about 500 ppm to about 10,000 ppm is most suitable in providing antimicrobial protection to the treated article.

For an article manufactured in whole or in part of metal, an antimicrobial powder coating technique can be utilized to affix a powder coat layer thereto. Powder coating of metal substrates is within the ken of the ordinarily skilled artisan.

Antimicrobial agents particularly suited for use in powder coating include silver, zinc, or copper in any of zeolitic, silica or silicate, zeolitic silicate, gel-sol, and similar forms that permit the metal to function in an ion-exchange manner without need for migration of the antimicrobial agent carrier vehicle within the powder coated layer.

While the amount of each antimicrobial agent may vary to some extent, the amount of each antimicrobial agent must be an amount sufficient to enable the treated component to exhibit an antimicrobial effect. That is, the treated article should demonstrate an ability to kill or suppress the replication of fungi, to kill or inhibit the growth of bacterial microorganisms, and/or to kill or suppress the replication of viruses.

This amount may well be different for each specific antimicrobial agent and it is within the skilled artisan's ability to relatively easily determine an effective concentration for each antimicrobial agent in a coating powder composition. In addition, an upper limit of the concentration of the antimicrobial agent may exist under certain circumstances such as when the solubility limit of the antimicrobial agent is reached.

Although the present invention has been described in considerable detail with reference to certain embodiments, one skilled in the art will appreciate that the present invention can be practiced by other than the described embodiments, which have been presented for purposes of illustration and not of limitation. Therefore, the scope of the disclosed invention should not be limited to the description of the embodiments contained herein.

1. A medical bed, comprising:
a bed frame having at least one frame member selected from the group consisting of a headboard, a footboard, a side rail member, and a support member; and
an antimicrobial agent incorporated in or disposed on the frame member.

2. The medical bed of claim 1 wherein the frame member is a structural member.

3. The medical bed of claim 1 wherein the antimicrobial agent is disposed in a material from which the frame member is constructed.

4. The medical bed of claim 1 wherein the antimicrobial agent is affixed to a material from which the frame member is constructed.

5. The medical bed of claim 1 wherein the antimicrobial agent is at least one of 2,4,4′-trichloro-2-hydroxydiphenyl ether or other substituted or unsubstituted phenol; or a biguanide compound.

6. The medical bed of claim 1 wherein the antimicrobial agent is a quaternary ammonium compound or a gentian violet compound.

7. The medical bed of claim 1 wherein the antimicrobial agent is an iodine compound, a silver compound, a copper compound, a zinc compound, or a mixture thereof.

8. The medical bed of claim 4 wherein the frame member has a powder coating layer affixed thereto, and the powder coating layer has incorporated therein the antimicrobial agent.

9. The medical bed of claim 8 wherein the antimicrobial agent is 2,4,4′-trichloro-2-hydroxydiphenyl ether or other substituted or unsubstituted phenol, an iodine compound, a silver compound, a copper compound, a zinc compound, or a mixture thereof.

10. The medical bed of claim 4 wherein the frame member has affixed thereto a frame cladding component having the antimicrobial agent incorporated in or disposed thereon.

11. The medical bed of claim 10 wherein the frame cladding component is made substantially of a polymeric material.

12. The medical bed of claim 11 wherein the polymeric material is an olefin.

13. The medical bed of claim 11 wherein the polymeric material is acrylonitrile butadiene styrene.

14. The medical bed of claim 11 wherein the antimicrobial agent is at least one of 2,4,4′-trichloro-2-hydroxydiphenyl ether or other substituted or unsubstituted phenol; or a biguanide compound.

15. An antimicrobial medical bed, comprising:
a frame element of a polymeric material; and
an antimicrobial agent incorporated within the polymeric material.

16. The antimicrobial medical bed of claim 15 wherein the polymeric material is acrylonitrile butadiene styrene or an olefin and the antimicrobial agent is 2,4,4′-trichloro-2-hydroxydiphenyl ether or other substituted or unsubstituted phenol, an iodine compound, a silver compound, a copper compound, a zinc compound, or a mixture thereof.
